(19) United States
(12) Patent Application Publication
(10) Pub. No.: US 2007/0249039 A1
(43) Pub. Date: Oct. 25, 2007

(54) MINUTE CREATURE DETECTION SYSTEM AND ANALYTIC CHIP

(76) Inventors: Mitsuhiro Matsuzawa, Kyoto (JP); Hisao Inami, Matsudo (JP); Yasuhiko Sasaki, Tsuchiura (JP); Masahiro Kurihara, Yokohama (JP)

Correspondence Address: ANTONELLI, TERRY, STOUT & KRAUS, LLP, 1300 NORTH SEVENTEENTH STREET, SUITE 1800, ARLINGTON, VA, 22209-3873, US

(21) Appl. No.: 11/627,402

(22) Filed: Jan. 26, 2007

(30) Foreign Application Priority Data
Apr. 25, 2006 (JP) 2006-120032

(51) Int. Cl. C12M 1/34

(52) U.S. Cl. 435/287.3

(57) ABSTRACT

It is an object of the present invention to shorten the operation time from sampling through to genetic test, and enable the detection of a minute creature with good accuracy. Provided is minute creature detection system in which treatments from gene extraction to detection of are conducted on an analytic chip, in which the analytic chip 300 contains a sample reservoir 315, a gene extraction area 320 filled with a gene binding carrier, a waste solution chamber 330 filled with absorbent, a washing solution storage chamber 340 which stores washing solution, an eluenting solution storage chamber 370 which stores a gene eluenting solution, a gene-amplification reagent storage chamber 380 which stores a gene-amplification reagent, and a reaction chamber 395 which amplifies and detects the gene, each of which is formed by a channel, and a weir having a channel in at least either the sample reservoir 315 or the reagent storage chamber 380 which contracts and expands.

### FIELD OF THE INVENTION

The present invention relates to a minute creature detection system and analytic chip which samples minute creatures floating in the air, and extracts genes in the minute creatures to conduct genetic examination of the minute creatures.

### BACKGROUND OF THE INVENTION

To provide an inexpensive chip having easy handling, and which enables the process from the extraction of a gene from a sample to the analysis of the gene to be automated in a batch, JP-A-2005-65607 describes the use of a gene-treating chip which comprises an injection port from which the sample containing the gene is fed, a lysis solution section which stores a lysis solution for introducing into the sample fed into the injection port, a gene-extracting section to which a liquid containing the sample and the lysis solution is introduced and which has a gene binding carrier which binds to the gene, a washing solution storage section which stores a washing solution for introducing into the gene-extracting section, an eluting solution storage section which stores an eluting solution for introducing into the gene-extracting section, and a reaction section to which the gene eluted by the eluting section is introduced.

### BRIEF SUMMARY OF THE INVENTION

The gene-treating chip used in the analyzer described in the above conventional technique is configured so that the necessary reagents for the gene treatment are already stored in the chip. However, because no consideration is given to stopping the reagents in a given location, the reagents may accidentally leak when carrying the gene-treating chip or when setting onto the analyzer. For this reason handling of the gene-treating chip is troublesome. In addition, as a consequence of accidental leakage, gene-treating operation, such as fluid control and the like, is unstable, which may lower detection accuracy.

It is an object of the present invention to solve the above-described problems of the conventional art, by enabling the operation from sample through to genetic test to detect minute creatures in a short time with good accuracy.

To solve the above-described problems, the present invention provides a minute creature detection system in which, a sampling chip is placed in a sampler to sample minute creatures on a sampling material and is then removed from the sampler and placed in an analyzer, and the sampling chip and a part of the solution treated by the sampling chip are moved to an analytic chip, and the treatments from gene extraction to detection in the analytic chip are conducted by liquid transport means of the analyzer, the analytic chip comprising a sample reservoir, a gene extraction area filled with a gene binding carrier, a waste solution chamber filled with absorbent, a washing solution storage chamber which stores washing solution, an eluenting solution storage chamber which stores a gene eluenting solution, a gene-amplification reagent storage chamber which stores a gene-amplification reagent, and a reaction chamber which amplifies and detects a gene, each of which is formed by a channel, and a weir having a channel in at least either the sample reservoir or the reagent storage chamber which contracts and expands.

Other objects, features and advantages of the invention will become apparent from the following description of the embodiments of the invention taken in conjunction with the accompanying drawings.

### BRIEF DESCRIPTION OF THE DRAWINGS

FIG. 1 is a flowchart illustrating the detection methods of bacteria according to one embodiment of the present invention;

FIG. 2 is a diagram illustrating the structure of a bacteria detection system according to one embodiment of the present invention;

FIG. 3 is an oblique perspective view of the sampler according to one embodiment;

FIG. 4 is a perspective view illustrating the method of mounting the sampling chip onto the sampler of FIG. 3;

FIG. 5 is a front view illustrating the sampling chip according to one embodiment;

FIG. 6 is a cross-sectional view of the sampling chip of FIG. 5;

FIG. 7 is a front view of an analytic chip according to one embodiment;

FIG. 8 is a cross-sectional view taken along the line A-A′ of FIG. 7;

FIG. 9(a) is an enlarged view of area B of FIG. 7; and FIG. 9(b) is a diagram illustrating the relationship between the contact angle and surface tension in the weir;

FIG. 10 is a diagram illustrating the structure of the main sections of an analyzer according to one embodiment of the present invention;

FIG. 11 is a diagram illustrating the cross-sectional structure of the analyzer in FIG. 10;

FIG. 12 is a diagram illustrating the structure of the substrate of the analyzer in FIG. 10; and

FIG. 13 is a diagram showing a profile of liquid handling according to one embodiment of the present invention.

### DESCRIPTION OF REFERENCE NUMERALS

100 Sampler
110 Lid
120 Nozzle
121 Primary filter
130 Chip support
140 Secondary filter
150 Support plate
160 Fan motor
170 Exhaust port
180 Controller
181 Display
185 Battery
190 Casing
191 Gripping section
200 Sampling chip
201 Sampling material
210 Germination promoter storage chamber
220 Enzyme A storage chamber
230 Enzyme B storage chamber
240 Chaotropic storage chamber
250 Air opening
300 Analytic chip
310 Sample injection port
311, 321, 331, 341, 351, 361, 371, 381, 391, 399 Chip port
315 Sample reservoir
320 Gene extraction area
330 Waste solution chamber
340 Washing solution I storage chamber
350 Washing solution II storage chamber
360 Washing solution III storage tan
370 Eluenting solution storage chamber
380 Gene-amplification reagent I storage chamber
390 Gene-amplification reagent II storage chamber
395 Reaction chamber
398 Liquid reservoir
400 Analyzer
401 Fore lid
402 Device internal channel
412 Substrate channel

### DETAILED DESCRIPTION OF THE INVENTION

The examples of the present invention will be described below.

An example will now be explained for detecting whether a bacteria of interest is present by sampling bacteria which have formed spores from the air, extracting a gene from the cell after the spores have been treated, and then amplifying the gene by a polymerase chain reaction, wherein the bacteria which form spores include genus Bacillus bacteria, genus Clostridium bacteria and the like.

Flow of Bacteria Detection:

Bacteria detection can be broadly classified into the steps of sampling the bacteria, germinating the bacteria spores by adding a germination promoter to the bacteria spores, extracting a gene from the germinated bacteria, and amplifying and detecting the gene. Gene extraction is conducted by a well-known solid-phase extraction method. A “solid-phase extraction method” is a method wherein a gene is specifically bound to a solid surface and then only the gene is eluted into an aqueous solution to differentiate from other substances.

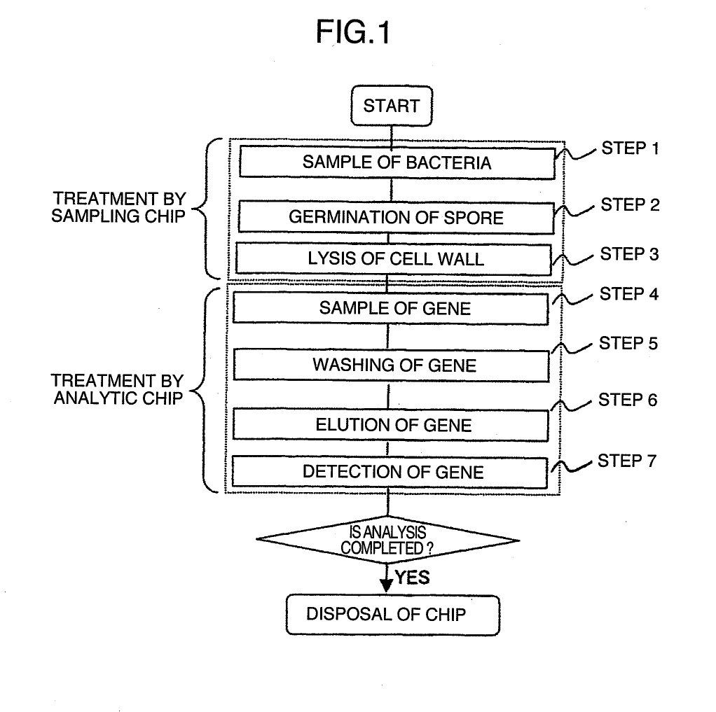

Referring to FIG. 1, a bacteria detection method will now be described.

### Step 1. Sample of Bacteria by Impactor Method:

An “impactor method” samples bacteria on an impactor plate provided below a nozzle by sucking air from above the nozzle and blowing it out the bottom of the nozzle at high speed. The bacteria in the air attain an inertia force in proportion to the square of their particle size, and adhere to the impactor plate. This method has the advantages of not causing clogging as in a filter method, and that the bacteria are gathered in a concentrated manner.

### Step 2. Germination of Spore:

A germination promoter is added to the bacteria spores, and after a certain amount of time has passed the bacteria spores start to germinate. At the germination stage, since the bacteria destroy the spores by themselves, the cell walls of the bacteria are in a naked state from germination.

### Step 3. Lysis of Cell Membrane:

A solution containing chaotropic ions (−1 negative ions having a large molecular diameter) is mixed into the sample, whereby the bacteria cell membranes are disrupted by the action of the chaotropic ions. The chaotropic ions also simultaneously denature a large amount of the proteins contained in the sample, whereby nuclease (an enzyme which breaks down nucleic acids) action is inhibited.

### Step 4. Sample of Gene:

If silica is added into the dissolved mixture, the gene and the silica are specifically bound from the action of the chaotropic ions. Typically, a method is used which passes the mixture through a glass filter.

### Step 5. Washing of Gene:

If proteins or chaotropic ions contained in the sample are mixed into the eluted product, detection of the gene by gene amplification is hindered. Thus, an operation to wash the gene-silica is required. This is normally conducted using highly concentrated ethanol. Since genes are hardly soluble in such solutions, the gene adsorbed on the silica does not elute in this process.

### Step 6. Elution of Gene:

After the washing, water or a low salt concentration solution is added to the gene-silica to elute the gene from the silica.

### Step 7. Detection of Gene:

The eluted gene is charged with a primer (single strand DNAs each having the same base sequence as a sequence of about 20 bases at each end of the intended DNA region), DNA synthetases (polymerases), four types of substrate (dNTP) and the like. By applying the temperature cycle “heat-denaturing—annealing—complementary strand synthesis”, the gene is amplified (polymerase chain reaction). Here, gene amplification can be detected in real time by pre-injecting with a fluorescent dye in addition to the above-described reagent and applying the temperature cycle while irradiating with excitation light.

Structure of Analysis System:

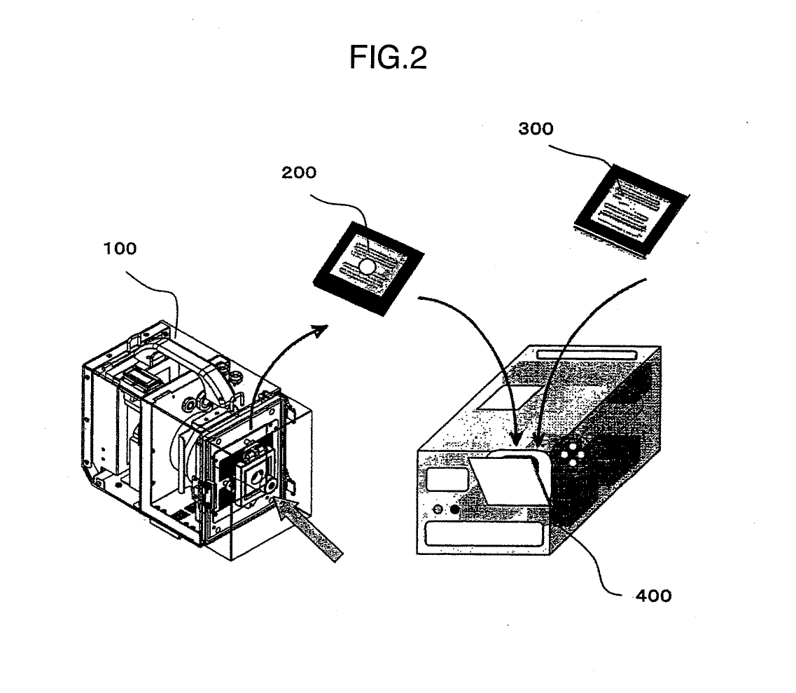

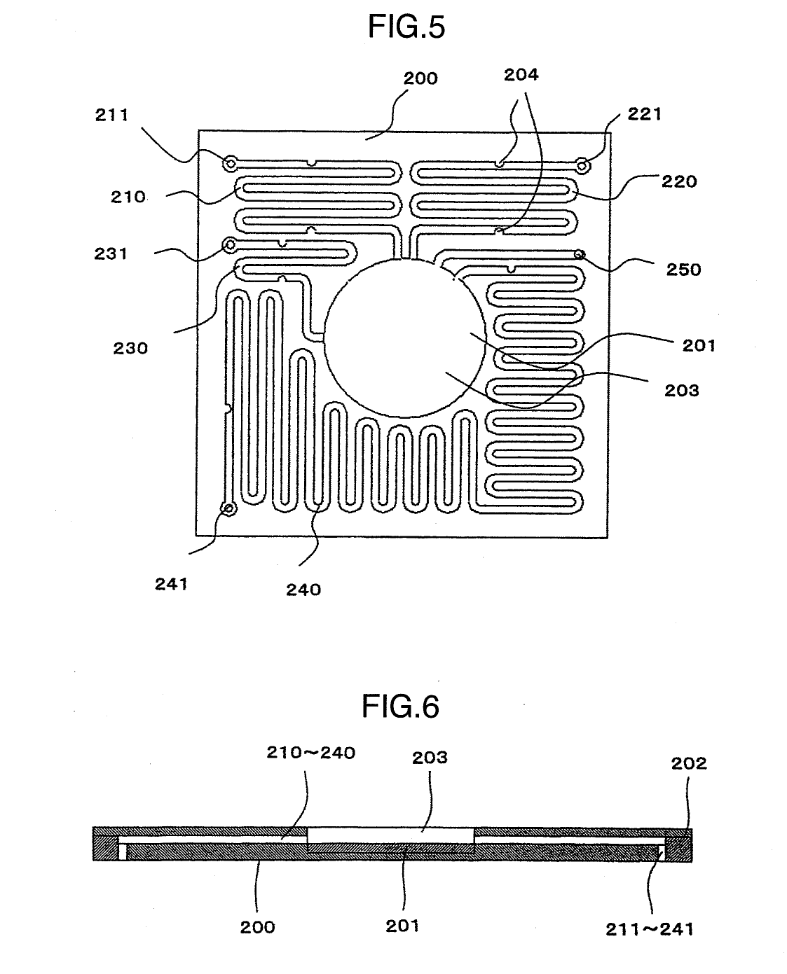

With reference to FIG. 2, the structure of an analysis system will be explained, which is comprised from the four components of a sampler 100, a sampling chip 200, an analyzer 400 and an analytic chip 300. Bacteria floating in the air are sucked in by the sampler 100 for sample on a sampling material 201 (refer to FIGS. 5 and 6) of the sampling chip 200 mounted in the sampler 100. The sampling chip 200 is removed from the sampler 100, and the aperture of the sampling material housing section 203 (refer to FIGS. 5 and 6) of the sampling chip 200 is blocked with a sealant. Alternatively, the sampling chip 200 is removed from the sampler 100 after the aperture of the sampling chip 200 has been blocked with a sealant, and is then placed in the analyzer 400. Thus, the sampling chip 200 having the above-described aperture sealed by the sealing material is placed in the analyzer 400 with minute creatures adhered to the sampling material. By handling the sampling chip 200 aperture when it is blocked with a sealing material, the sampling chip 200 can be handled safely. The analyzer 400 is equipped with liquid transport means. Reagent in the sampling chip 200 is transported with the sampling chip 200 placed in the analyzer 400. Germination of the bacteria spores and lysis of the cell membranes are performed in the sampling chip 200.

Bacteria are sampled using the sampling chip 200 which has been pre-embedded with a plurality of reagents. The sampling chip 200 which has sampled the bacteria will be used as-is for the next several minute creature detection treatments, which makes it convenient to use from sample to minute creature detection treatment.

The sampling chip 200 is subsequently removed from the analyzer 400, and a part of the liquid processed by the sampling chip 200 is moved to the analytic chip 300 (since the bacteria have already been disrupted by lysis, they cannot cause contamination even if touched).

The analytic chip 300 is placed in the analyzer 400. The analytic chip 300 is placed in the same location as the sampling chip 200 had been placed. The analytic chip 300 is pre-embedded with the reagents for Step 4 (Sample of Gene) to Step 7 (Detection of Gene) of FIG. 1. With the analytic chip 300 placed in the analyzer 400, the reagent in the analytic chip 300 is transported by the liquid transport means of the analyzer 400, whereby gene extraction through gene detection is performed in the analytic chip 300. Once the analysis is completed, the analytic chip 300 is removed from the analyzer 400 and discarded.

The reagents necessary for the steps from pre-processing through to detection of the bacteria are all embedded in two kinds of minute creature detection chips (sampling chip 200 and analytic chip 300), whereby cumbersome reagent operations can be obviated. The present invention is very safe, since apart from the step of transferring the sample between the two kinds of chip 200 and 300, the sample does not leave the chip, whereby analysis is conducted in a closed system. Further, the only discarded products are the chips 200 and 300, so that if these chips 200, 300 are made from a material which can be incinerated, the risk of secondary contamination can be reduced.

In addition, the reagent embedded in the two kinds of chip 200, 300 are sufficient for only one detection, and thus the chips 200, 300 can be employed as single use chips, which enables high-accuracy bacteria detection at the gene level to be performed simply outdoors.

The two kinds of chip 200, 300 embedded with the reagent are categorized into the chip 200 which conducts processing from sample of the bacteria through to lysis, and the chip 300 which conducts the analytical processing thereafter. This means that it is easy to conduct multiple analyses using a sample having the same processing from sample through to lysis, whereby improvement in accuracy can be easily achieved while maintaining safety.

Structure of Sampler:

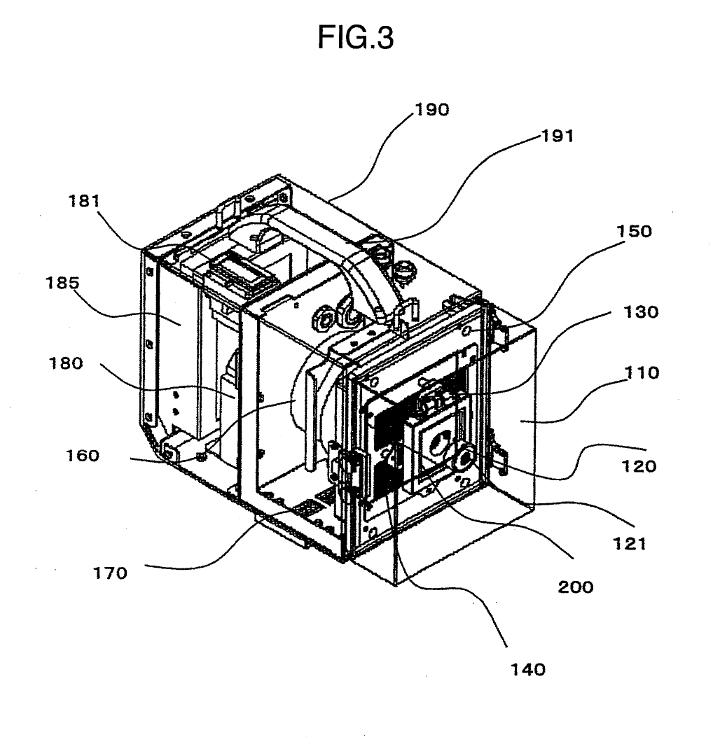

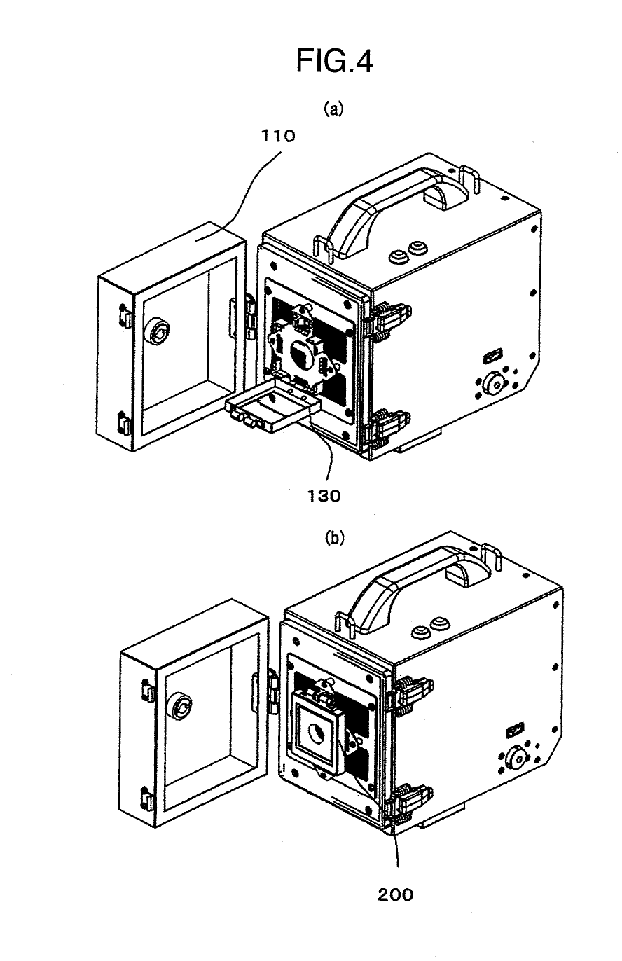

FIG. 3 is an oblique perspective view of the sampler 100. FIG. 4a is a diagram illustrating a chip support 130 with the lid 110 of the sampler 100 open. FIG. 4b is a diagram illustrating a sampling chip 200 mounted with the chip support 130 closed.

The sampler 100 comprises a lid 110, a nozzle 120, a primary filter 121, a chip support 130, a secondary filter 140, a support plate 150, a fan motor 160, an exhaust port 170, a controller 180, a display 181, a battery 185 and a casing 190. The lid 110 is a square or rectangular section comprising the nozzle 120 and has fixing means on either side.

The inner diameter of the nozzle 120 has a strong bearing on sample efficiency. While bacteria can be sampled in a more concentrated manner if the nozzle inner diameter is smaller than 10 mm, pressure loss increases as a result of the air flow rate that is flowing through the nozzle 120 increasing. The pressure loss increases in square proportion to the air flow rate, thereby increasing the load on the fan motor 160 and decreasing the current of the battery 185. For example, if the inner diameter of the nozzle 120 is 3 [mm] or less, the drivable load by the battery 185 (lithium-hydrogen) which can be mounted on the portable bacteria sampler 100 is exceeded. Therefore, the inner diameter W of the nozzle 120 is preferably 4 to 15 [mm], and more preferably 8 to 12 [mm]. In this range bacteria can be concentrated for sample directly underneath the nozzle 120 while achieving a high sample efficiency.

A primary filter 121 is mounted on the nozzle 120. The primary filter 121 is provided in order to trap coarse particles in the air. Accordingly, its apertures are preferably between 100 and 200 μm. During periods when there is a large amount of pollen in the air, the apertures are preferably between 10 and 100 μm. Within this range, pollen having a particle size of 10 μm or more and bacteria having a particle size of less than 10 μm can be easily separated. The primary filter 121 is detachable from the lid 110, and is preferably made from stainless steel or a synthetic resin which is produced by polymerizing tetrafluoroethylene, which is easily washed and sterilized at high temperatures.

The chip support 130 is mounted on (front) the secondary filter 140. The chip support 130 is retractable (FIG. 4), and if the nozzle 120 is gripped and the lid closed, the sampling chip 200 can be easily placed in the sampler 100. Since the chip support 130 corresponds to the “periphery” of the sampling chip 200, bacteria are easily adhered. Therefore, the chip support 130 is preferably detachable from the secondary filter 140, and made from stainless steel or a fluororesin, which are easily washed and sterilized at high temperatures.

The secondary filter 140 is mounted on the support plate 150. The secondary filter 140 is provided in order to prevent bacteria fine particles which could not be sampled by the sampling chip 200 from being released into the air. The secondary filter 140 preferably employs a HEPA (High Efficiency Particulate Air) filter which can sample at least 99.97% of fine particles having a size of 0.3 μm or more.

More preferably, a ULPA (Ultra Low Penetration Air) filter is employed which can sample at least 99.999% of fine particles having a size of 0.1 to 0.2 μm. By using a ULPA filter, the cleanliness of the air released from the exhaust port 170 into the ambient atmosphere can be further increased. The controller 180, display 181 and battery 185 are provided in the casing 190. On an upper face of the casing 190 is provided a gripping section 191.

Next, operation of the sampler 100 will be described. If the fan motor 160 is activated, air is sucked into the nozzle 120. The suctioned air is accelerated by the nozzle, and passes through the primary filter 121. Coarse particles in the air are removed at this point by the primary filter 121. The fine particles in the air which has been introduced into the lid 110 are adhered by inertial collision with the sampling material provided in the center of the sampling chip 200. The air introduced into the lid 110 passes through the secondary filter 140, and is externally exhausted from the exhaust port 170 provided on a lower portion of the fan motor 160. Fine particles which were not sampled on the sampling chip 200 are removed by the secondary filter 140.

Structure and Operation of Sampling Chip:

An example of the sampling chip 200 will now be described with reference to FIGS. 5 and 6. FIG. 5 is a front view of a sampling chip 200, and FIG. 6 is a cross-sectional view of the sampling chip 200 along its length.

The sampling chip 200 handles from Step 1 (Sample of Bacteria) to Step 3 (Lysis of Cell Membrane) of FIG. 1. Specifically, once the sampling chip 200 has been placed in the sampler 100 and bacteria have been sampled (Step 1), the sampling chip 200 is removed from the sampler 100 and placed in the below-described analyzer 400, and then the processes from Step 2 (Germination of Spore) and Step 3 (Lysis of Cell Membrane) are conducted.

The sampling chip 200 is prepared with a pattern which models the structural elements of the chip by a photolithography technique. This pattern is a cast which is copied onto the resin by molding. Most of the patterns are fine channels. Thus, by sticking together two sheets of resin, the patterns carved into the resin form a channel. As the chip material, it is preferable to use a resin, which has excellent disposability, rather than glass which is expensive to work and is easily broken. The kind of resin is not especially limited, but polydimethylsiloxane having the following excellent characteristics is used, so that the chip preferably comprises the following characteristics:

Excellent biocompatibility (ordinary silicone rubber is physiologically inert)

Copying of pattern can be effected with submicron precision (before curing, it has low viscosity and high fluidity, and thus can favorably permeate into the intricacies of complicated shapes)

Low cost (8 yen/gram. Not more than a one-hundredth of that of conventional general-purpose material, Pyrex® glass, for microdevices which is 1000 yen/gram)

Easily disposable by incineration

The sampling chip 200 comprises the sampling material 201 on which minute creatures (bacteria which have formed spores) from the air have adhered for sample, and a thin plate-shaped substrate 202 mounting the sampling material 201. The sampling chip 200 has a sampling material housing section 203 for housing the sampling material 201, a plurality of reagent storage chambers (210, 220, 230, 240), chip ports 211, 221, 231, 241 which are open to the chip rear face, and an air opening 250 which is open to the chip front face.

The reagent storage chambers comprise a germination promoter storage chamber 210 which stores a germination promoter, an enzyme A storage chamber 220 which stores two kinds of cell wall lysis solution, an enzyme B storage chamber 230, and a chaotropic storage chamber 240 which stores chaotropic ions. The plurality of reagent chambers 210 to 240 are provided so as to encompass the periphery of the sampling material housing section 203, thereby enabling the substrate 202 to be made compact.

The reagent chambers 210 to 240 are configured from long, thin channels, and it is preferable for all of the reagent chambers to be in a channel shape. To transport reagent to the reagent chambers 210 to 240, gas is sent from the rear side of the reagent chambers 210 to 240 to the reagent chambers 210 to 240. If the reagent chambers 210 to 240 are formed in a long, thin channel shape, reagent is only pushed out of locations where the gas can easily pass through, meaning that reagent remains in the reagent chamber at the other locations. To lower the amount of reagent being consumed, it is effective for the reagent chambers 210 to 240 have a channel shape. The cross-sectional shape of a channel preferably is not especially limited, but has a width to length ratio of not greater than 10. If the width to length ratio is greater than 10, the resin of the channel ceiling may sag thereby destroying the rectangular shape of the channel. The long, thin channel of the reagent chambers 210 to 240 is formed in a serpentine shape, thereby ensuring storage capacity of the reagent in the channel while reducing the area that the channels occupy on the substrate 203.

One end of the reagent chambers 210 to 240 is connected to the sampling material housing section 203, and the other end of the reagent chambers 210 to 240 is connected in communication with a chip port (211 to 241). The one end of the reagent chambers 210 to 240 and the other end of the reagent chambers 210 to 240 are each provided with a weir 204, whereby the flow of the reagent stored in each of the reagent chambers 210 to 240 can be reliably stopped. The chip ports 211 to 241 form junctions with the external channels. The germination promoter storage chamber 210, enzyme A storage chamber 220, enzyme B storage chamber 230 and chaotropic storage chamber 240 are all in communication with the sampling material housing section 203. Therefore, the sampling material housing section 203 is connected to external channels via the germination promoter storage chamber 210, enzyme A storage chamber 220, enzyme B storage chamber 230 and chaotropic storage chamber 240 and the chip ports 211 to 241. By narrowing the channel width of the reagent chambers 210 to 240 to 50 to 100 μm, the influx of air can be prevented from the sampling material housing section 203 side.

The volume of the germination promoter storage chamber 210 is preferably 20 to 100 μL, the volume of the enzyme A storage chamber 220 is preferably 20 to 100 μL, the volume of the enzyme B storage chamber 230 is preferably 5 to 20 μL, and the volume of the chaotropic storage chamber 240 is preferably 400 to 800 μL. Disruption of the cell membranes is promoted by making the volume of the chaotropic ions at least twice the total volume of the two kinds of germination promoter. More preferred is 4 times or greater, and optimally, 8 times or greater.

Agar is preferred as the sampling material 201. The characteristic of the agar is that it has free water on the gel surface (water among the gel network). The agar concentration is preferably 2 to 5%, and most preferably 3 to 4%. If the agar concentration is less than 2%, the moisture content is large, meaning that its strength as the sampling material 201, on which high-speed air is continuously applied, is insufficient. On the other hand, if the agar concentration is greater than 6%, the moisture content (free water) of the agar surface is reduced, whereby adhesion dramatically decreases.

To increase the strength of the agar and stop the moisture from evaporating, it is effective to add an alcohol, which acts as an antifreeze agent, drying-prevention agent and a gel strengthening agent. Specific examples thereof include ethyl alcohol, isopropyl alcohol, 1,3-butandiaol, ethylene glycol, propylene glycol, glycerin and the like. The added amount of alcohol is preferably 40 to 80% of the agar, and more preferably 50 to 70%. If the added amount is less than 40%, evaporation prevention of moisture is insufficient, and if the added amount is more than 80%, the moisture content (free water) of the agar surface is reduced, whereby adhesion decreases.

One example of a method for using the sampling chip 200 will now be described. The sampling chip 200 is attached to the chip support of the sampler 100 so that air is sucked in for a certain period of time. The amount of suctioned air is, for example, about 1,000 L. Bacteria in the air adhere to the sampling material 201 surface of the sampling chip 200. Next, the sampling chip 200 is taken off the chip support. After the aperture surface of the sampling material housing section 203 of the sampling chip 200 has been sealed, the sampling chip 200 is placed in the analyzer 400. While sealing of the sampling material housing section 203 may be conducted manually, is preferred to have a sealing mechanism on the sampler. Sealing the sampling material housing section 203 prevents bacteria being externally exposed from the sampling chip 200, thereby improving safety.

The reagent chambers 210 to 240 of the sampling chip 200 are connected to external channels via chip ports (211 to 241). Thus, by feeding gas via the chip ports 211 to 241 by a predetermined control operation from the channel side of the analyzer 400, the germination promoter, cell wall lysis solution, cell membrane lysis solution and chaotropic ions encapsulated in the reagent chambers 210 to 240 are transported at predetermined times into the sampling material housing section 203 (onto the sampling material 201). Although the upper front face of the sampling material housing section 203 is sealed, because an air opening 250 is in communication with a part of the sampling material housing section 203 and thus open to the air, when transporting the reagent air present above the sampling material 201 is released from the air opening 250.

Details of the transportation of the reagent from the reagent chambers 210 to 240 to the sampling material 201 will now be described.

100 μL of the germination promoter is transported to the sampling material 201. Here, the germination promoter is preferably a broth comprising alanine, adenosine or glucose. A broth comprising 1 mM to 10 mM of L-alanine is especially preferable. After 10 minutes have passed, germination of the bacteria spores starts, and after 30 minutes have passed, at least 50% have germinated. Therefore, the spore germination treatment is preferably not less than 30 minutes. A preferable temperature to cause the bacteria spores to germinate is 35 to 40° C., and most preferred is 35 to 37° C. Since the bacteria destroy the spores by themselves during the stage wherein the bacteria spores germinate, the cell walls of the bacteria are in a naked state from germination.

Next, the two kinds of protein-modifying enzyme are successively transported to the sampling material 201, and an optimum temperature is maintained for a certain period of time. The duration of the enzyme treatments is preferably respectively 10 minutes or more, and 30 minutes is suitable. Here, as the protein-modifying enzyme, 100 μL of a lysozyme (an optimum temperature of 37° C.) and 20 μL of protease K (an optimum temperature of 55 to 60° C.) is suitable. As a result of these enzyme treatments, the bacteria in the sampling chip 200 have their cell membrane bared. Further, while the germination promoter and the lysozyme can be introduced at the same time, the lysozyme and the protease K should not be added at the same time, as this reduces enzyme activity.

Finally, by transporting 800 μL of chaotropic ions to the sampling material 201, the bacteria cell membranes are disrupted, whereby a bacteria gene is released out of the cells. Examples of chaotropic ions include guanidine thiocyanate, guanidine hydrochlorate, sodium iodide, potassium bromide and the like. Examples of the method for using the chip include maintaining the activity of the reagents for a long period of time by refrigerating or freezing the chip. Therefore, guanidine hydrochlorate is preferable, as it shows very little change in structure when refrigerated or frozen.

It is also preferable to incorporate a surfactant or a buffering agent in the chaotropic salt. Preferable examples of the buffering agent include tris-hydochloride, potassium dihydrogen phosphate/sodium tetraborate and the like.

According to the above steps, germination of the cells sampled on the sampling chip 200 and treatment of the cell walls can be conducted. Specifically, as the operation can be automated on the chip as far as Steps 2 or 3 illustrated in FIG. 1, it is no longer necessary to dispense the reagents separately. The sampling chip 200 handles the steps from the sample of bacteria to pretreatment, and the analytic chip 300 handles the step of analyzing the bacteria gene. To increase the accuracy of the analysis, it is preferable to analyze the same sample multiple times. Alternatively, to set a plurality of target bacteria, it is preferable to divide a sample treated by a single sampling chip 200 among plural analytic chips 300, so that two kinds of chip 200, 300 are provided.

It is noted that if the sampling chip 200 is given to a user while frozen and the user stores the analytic chip 300 frozen at 0° C., the activity of the reagent can be maintained for one month. In addition, by storing frozen at −20° C., the activity of the reagent can be maintained for half a year or longer.

Structure and Operation of Analytic Chip:

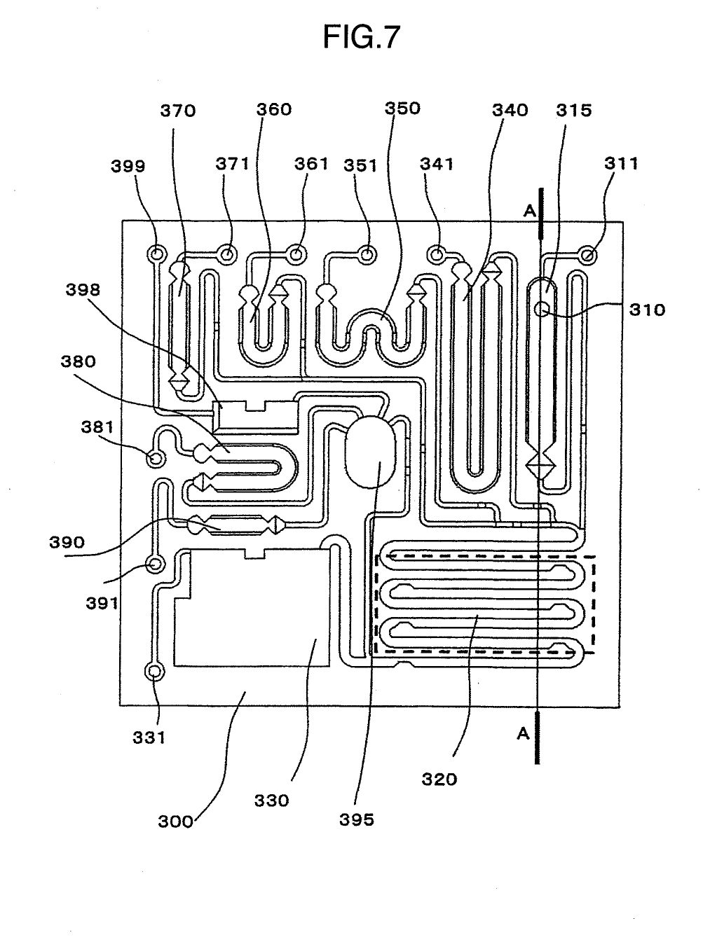

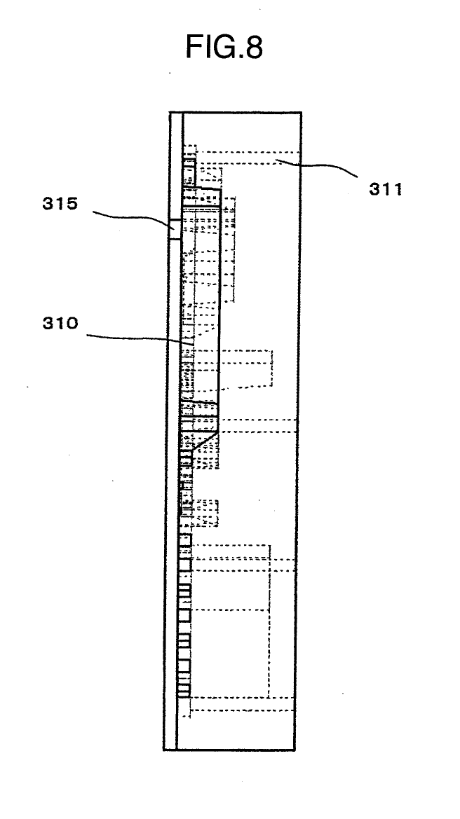

The analytic chip 300 will now be specifically described with reference to FIGS. 7 to 9.

The analytic chip 300 handles from Step 4 (Sample of Gene) to Step 7 (Detection of Gene) of FIG. 1. A part of the liquid treated by the sampling chip is placed in an analyzer 400 which has been moved. The reagents which will be used in Step 4 (Sample of Gene) to Step 7 (Detection of Gene) of FIG. 1 are pre-embedded in the analytic chip 300. With the analytic chip 300 placed in the analyzer 400, the liquid transport means on the analyzer 400 is activated to transport the reagents in the analytic chip 300, and the treatments from gene extraction to gene detection are conducted in the analytic chip 300. The material for the analytic chip 300 is the same resin as that of the sampling chip 200.

The analytic chip 300 comprises a sample injection port 310 open to the chip front face, a sample reservoir 315, a gene extraction area 320 filled with a gene binding carrier in the channel, a waste solution chamber 330, a washing solution I storage chamber 340 which stores a washing solution I, a washing solution II storage chamber 350 which stores a washing solution II, a washing solution III storage chamber 360 which stores a washing solution III, an eluting solution storage chamber 370 which stores a gene eluting solution, a gene-amplification reagent I storage chamber 380 which stores a gene-amplification reagent I, a gene-amplification reagent II storage chamber 390 which stores a gene-amplification reagent II, a reaction chamber 395 which amplifies and detects the gene, and chip ports 311, 331, 341, 351, 361, 371, 381 and 391 which are open to the chip rear face. A liquid reservoir 398 is provided between the reaction chamber 395 and the chip port 399. Providing the liquid reservoir 398 can prevent reagent from accidentally passing from the reaction chamber 395 through the chip port 399 and penetrating into the analyzer 400.

The cross-sectional shape of the reagent storage chambers 340 to 390 is preferably the same as that of the sampling chip; i.e. a width/length of not more than 10. If the width/length is more than 10, the resin of the channel roof may sag, thereby causing the channel to lose its rectangular shape. In the drawings, the width is 2 mm and the length is 3 mm.

The cross-section of the channels other than the reagent chambers 340 to 380 is preferably smaller than that of the reagent chambers 340 to 390, and a cross-sectional area of ¼ is preferable. The analytic chip 300 is fabricated by copying from a resin mold fabricated by a stereolithography. Since it is difficult to form a resin fabricated by a stereolithography with smooth curves, a rectangular structure is generally used. Thus, the channel cross-section of an analytic chip copied from a resin mold having a rectangular structure will naturally be rectangular structure. When a reagent is flowed through a rectangular channel having rectangular structure, reagent tends to adhere to the four corners of the channel and remain there. Since reagent remaining in the channel mixes with the reagent which is flowed through next, analysis accuracy deteriorates. Then, reagent adherence to the channel wall surfaces is suppressed and “carry over” of the reagent prevented by decreasing the cross-section of the channel. Specifically, the width is 0.5 mm and the length is 0.5 mm.

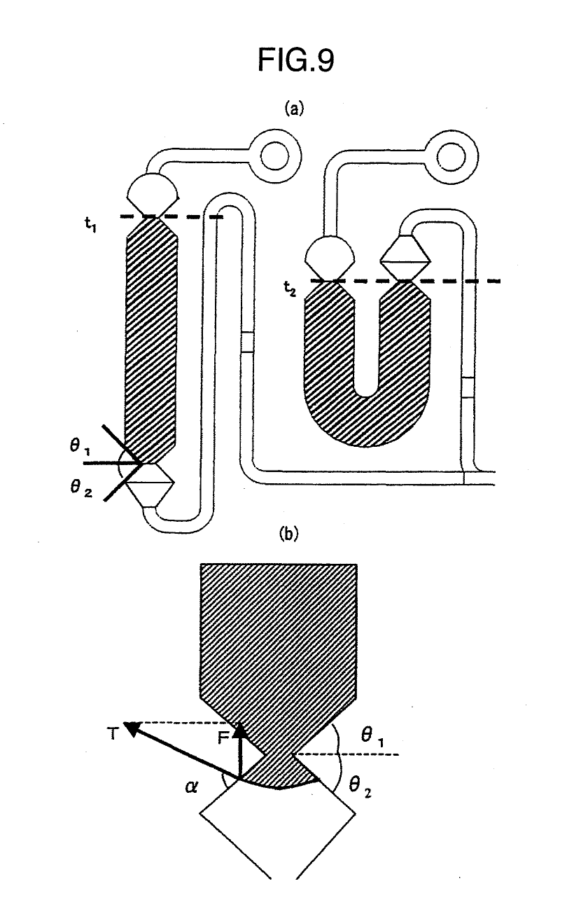

As illustrated in FIG. 9(a), the channel width of the sample reservoir 315 and at least one end of the reagent storage chambers 340 to 390 contracts, and a weir is provided as a section which again rapidly expands. FIG. 9(b) illustrates the relationship between the reagent in the weir and the surface tension acting on the weir. Taking the contact angle between the reagent and the channel as α, the angle of the contracted weir as θ₁, the angle of the expanded weir as θ₂, the surface tension of the reagent as T, the vertical component of the surface tension as F, the circumference length of the channel as L: F′=F×L. In this situation, the relationship among F, α, θ₁, θ₂ and T is:

F=T sin(α−θ₂)

Therefore, when F>0, i.e. α−θ₂>0, F′ acts as a suppressing force to stop the leakage of reagent, wherein the closer that α−θ₂ is to 90°, the greater F′ becomes. When α−θ₂ is 90°, the suppressing force for stopping the reagent is at a maximum. The following case will be used as an example to illustrate this. If the gene eluenting solution is water, and θ₂ is 45°, the contact angle α between the water and the PDMS is 85°, meaning that the smaller θ₂ is the greater F′ becomes. On the other hand, this also means that if θ₂ is small, liquid is more likely to remain. Here, if θ₂ is 45°, α−θ₂ is 40°. Since the surface tension T of water is 7.26×10⁻⁴ [N/cm] (20° C.), the vertical component is F=7.26×10⁻⁴×sin 40°=4.46×10⁻⁴ [N/cm]. If, for instance, the circumference of the constriction of the eluenting solution storage chamber is set as L=2×(0.5+3)=7 [mm], then the suppression force F′=4.67×10⁻⁴×7×10⁻¹=3.26×10⁻⁴ [N]. Taking the volume of the gene eluenting solution to be about 20 [μL], then the gravitational force acting on the gene eluenting solution is about 2.00×10⁻⁴ [N]. Accordingly, in this case, it appears that there is sufficient force to stop the flow of eluent due to gravitational force. The shape of the weir can be optimized considering the surface tension of the reagent, the contact angle between the reagent and PDMS, as well as the residual liquid. Providing the weir allows accidental leakage of the reagent to be stopped by utilizing surface tension.

As illustrated in FIG. 9(a), if the analytic chip 300 is used in an upright manner, the reagent can be stopped from accidentally leaking any further along a channel if the channels in communication with each of the reagent storage chambers are once arranged higher than the liquid surfaces t₁, t₂ of the estimated maximum liquid amount to be stored in each of the reagent storage chambers.

Accidental leakage of the reagent can be stopped with high probability without requiring a complex mechanism by combining a rapid expanding section of the channel with a channel structure providing the channel in communication with the reagent storage chamber higher than the liquid surface. Stopping accidental leakage of the reagent obviates the need for special care to be given in handling the chip and also improves the handling ease of the chip. In addition, fluid control of the reagent can be conducted more stably, and gene detection accuracy improves.

Further, since liquid is more likely to remain if the angle of contraction θ₁ is small, θ₁ is preferably between 10° and 80° (inclusive thereof). If the angle of expansion θ₁ is large, the suppressing force from the surface tension of the reagent decreases (when α−θ₁>0), or the propelling force from the surface tension increases (when α−θ₁<0). Thus, θ₁ is preferably not more than 80°. If the angle of expansion θ₂ is large, the propelling force from the surface tension also increases. Thus, θ₂ is preferably not more than 45°. Further, since liquid is more likely to remain if the expansion angle θ₂ is too small, θ₂ is preferably not less than 5°. While the present example is illustrated with the channel width contracting and expanding linearly, the width may also contract or expand in a non-linear manner. In the case of producing the chip by transferring the pattern with an optical mold, the length/width is preferably not more than 10. If length/width is more than 10, it is difficult to peel off the chip from the mold. In the case where it is difficult to peel off the chip from the mold even though length/width is not more than 10, it is preferable to provide a slight taper to the mold (e.g. a 5° taper) to make peeling off easier.

Chip ports are formed on one end of the sample reservoir 315, waste solution chamber 330 and reagent chambers 340 to 390. The chip ports form junctions with the channels of the external analyzer 400. To transport the reagent in the reagent chambers 340 to 390, gas from the analyzer 400 which is external to the chip is sent to the reagent chambers 340 to 390 via the chip ports. Since oxygen can oxidize the reagent, and carbon dioxide can change the pH of the reagent, preferable examples of the gas are inert gases, such as nitrogen, helium, argon or the like.

The sample reservoir 315, washing solution I storage chamber 340, washing solution II storage chamber 350, washing solution III storage chamber 360 and eluenting solution storage chamber 370 are all in communication with the gene extraction area 320. Mixing of the sample, the washing solution I or the washing solution II with the dissolving solution inhibits the detection of the gene, so that it is preferable to position the eluting solution storage chamber 370 away from the sample reservoir 315, the washing solution I storage chamber 340 and the washing solution II storage chamber 350.

The gene-amplification reagent I storage chamber 380 and the gene-amplification reagent II storage chamber 390 are in communication with the reaction chamber 395. Once the gene-amplification reagent I sent to the reaction chamber 395 from the gene-amplification reagent I storage chamber 380 and the gene-amplification reagent II sent to the reaction chamber 395 from the gene-amplification reagent II storage chamber have been introduced into the reaction chamber 395, the gene-amplification reagent I storage chamber 380 and the gene-amplification reagent II storage chamber 390 are preferably in communication through the top of the reaction chamber 395 to prevent reflux from the reaction chamber 395.

The sample reservoir 315 preferably has a volume of 100 to 200 μL, the gene extraction area 320 preferably has a volume of 100 to 200 μL, the washing solution I storage chamber 340 preferably has a volume of 200 to 300 μL, the washing solution II storage chamber 350 preferably has a volume of 50 to 150 μL, the washing solution III storage chamber 360 preferably has a volume of 30 to 100 μL, the eluenting solution storage chamber 370 preferably has a volume of 10 to 30 μL, the gene-amplification reagent I storage chamber 380 preferably has a volume of 20 to 40 μL and the gene-amplification reagent II storage chamber 390 preferably has a volume of 10 to 20 μL.

Examples of suitable materials which may be employed as the gene binding carrier filled into the gene extraction area 320 include quartz wool, glass wool, glass fiber and glass beads. If using glass beads, to increase the contact surface area, the size of the beads is preferably not more than 50 μm, and in consideration of blockages in the channel, 20 to 30 μm is most preferable.

Structure and Operation of Analyzer:

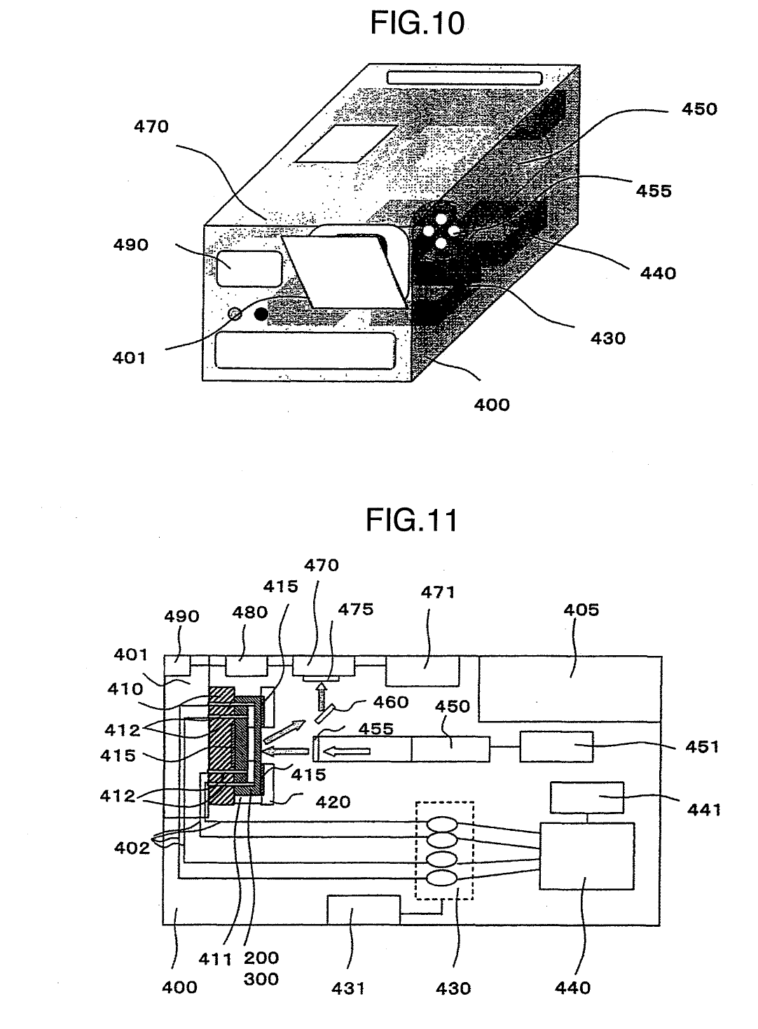

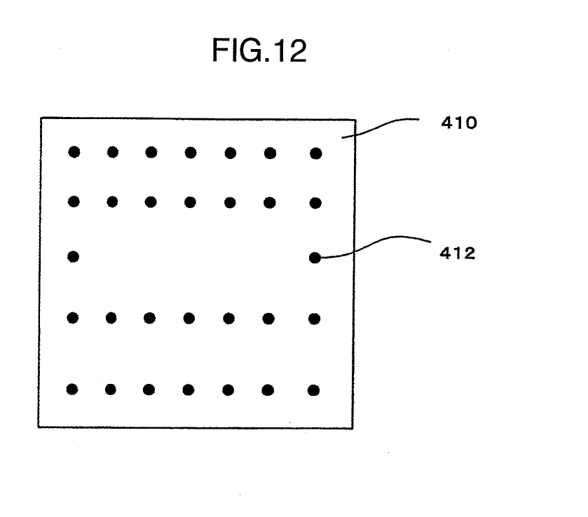

The structure and operation of the analyzer 400 according to the present invention will now be described with reference to FIGS. 10 to 13. FIG. 10 is a diagram illustrating the main structure of the analyzer 400; FIG. 11 is a diagram illustrating the cross-sectional structure of the analyzer 400; and FIG. 12 is a diagram illustrating the substrate 410 of the analyzer 400.

The analyzer 400 is roughly divided into four systems: a chip positioning section, a fluid system, a temperature control system, and an optical detection system. The substrate 410 on which the sampling chip 200 and analytic chip 300 are placed is provided on an inner side of the fore lid 401. In this example, since both chips are placed in an upright manner, a chip stopper 411 for stopping the chips is provided at a lower portion of the substrate 410. If the chips are placed on the substrate 410 and the fore lid 401 is closed, the chips are fixed in between the chip substrate 410 and the chip holder 420. The chip substrate 410 and the chip holder 420 are provided with a temperature control mechanism 415 for optimizing the temperature of the chips. While various heat generating bodies can be used as the temperature control mechanism 415, a Peltier device is a preferable example thereof. If a Peltier device is used, the heating/cooling operation can be easily conducted just by changing the direction of the applied current.

The substrate 410 is provided with a substrate channel 412. One end of the substrate channel 412 is in communication with a chip port, while the other end of the substrate channel 412 is in communication with a device internal channel 402. The substrate is provided in advance with a plurality of substrate channels 412, so that the substrate channel 412 can be applied to the chip ports of either the sampling chip 200 or the analytic chip 300, whereby the analyzer 400 can become a platform for the sampling chip 200 and the analytic chip 300.

The device internal channels 402 are respectively connected to a pump 440 via a valve 430. To transport the reagent in the reagent chamber in a given chip, the valve 430 is switched to blow only along the channel in communication with that reagent chamber. Thus, the gas sent by the pump 440 is delivered into the chip via the selected device internal channel 402 and substrate 410 channel, whereby the reagent in the reagent chamber is transported. Since a predetermined amount of reagent is pre-embedded in the reagent chamber, the reagent in the reagent chamber may be just discharged by time management, thus obviating the need for transport accuracy of the pump 440. Accordingly, the pump 440 only blows and does not suck, so that a simple and compact device may be used. The valve 430 that controls the fluid is preferably provided at the analyzer 400 side rather than inside the chip. By so doing, the chip 101 becomes free of mechanical parts, thereby attaining size-reduction and disposability.

The optical detection system is composed of a light source 450 that irradiates an excitation light onto the gene in the chip reaction chamber 390, an excitation filter 455 which only allows through excitation light having a specific wavelength, a mirror 460 which changes the light path of the fluorescence generated from the chip reaction chamber 390, a detection filter 475 which only allows through fluorescence having a specific wavelength, and a photodetector 470 which measures fluorescence. As the light source 450, while devices having different wavelength regions may be used, a xenon lamp is used which has a broad wavelength region. In cases where the wavelength is limited, an LED is desirably used. Examples of the photodetector 470 include a CCD camera, a photomultiplier tube, a photodiode and the like, with the photodiode being preferred in order to reduce the size of the device. Light signals of the gene detected by the photodetector 470 are digitized by a light signal converter 480, and the signal intensity is displayed on the data display screen 490.

The analyzer 400 is provided with control mechanisms for performing each control. On the analyzer 400 are mounted a valve control mechanism 431 which controls the valve 430, a pump control mechanism 441 which controls the pump 440, a light source control mechanism 451 which controls the light source 450, and a photodetector control mechanism 471 which controls the photodetector 470. Thus, a compact and portable analyzer can be provided by placing a compact analytic chip which is free from mechanical parts on the substrate, and combining with a simple photodetector.

Analysis Procedure:

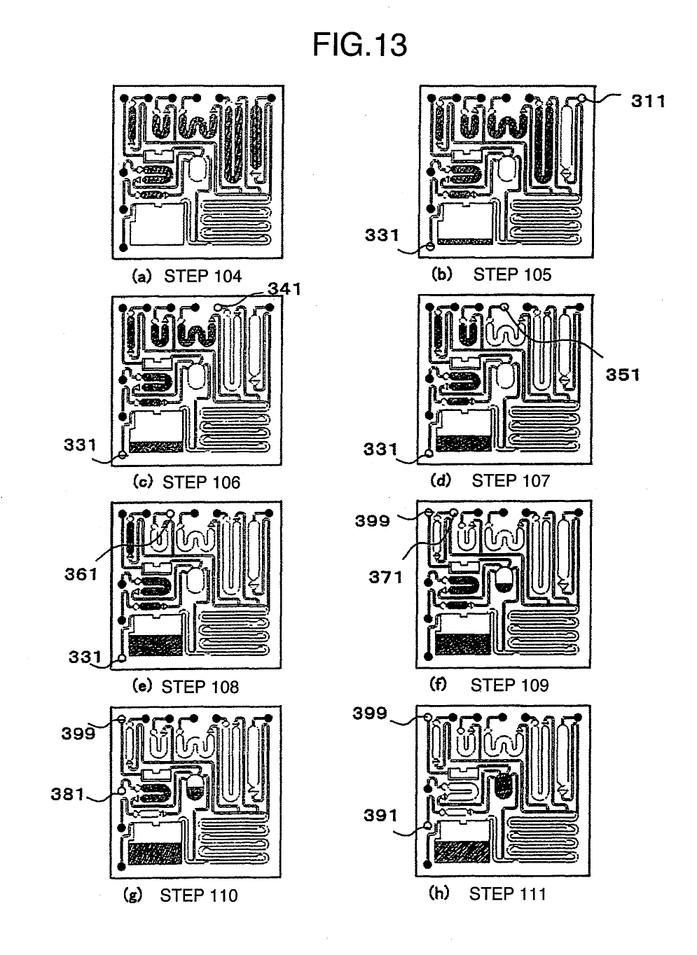

The analysis procedure using the analytic chip 300 and the analyzer 400 will now be described with reference to FIG. 7, FIG. 11, and FIG. 13. FIG. 13 is a diagram showing a profile of fluid handling of Example 1.

A bacteria sample whose cell walls have been dissolved by the sampling chip 200 is injected into the analytic chip 300. At the analytic chip 300, the bacteria sample is transported to a channel filled with a gene-retaining carrier. Then, a washing solution which washes proteins etc. contained in the sample is transported to the channel filled with the gene-retaining carrier.

Next, an eluting solution which elutes the gene adsorbed to the gene-retaining carrier is transported to the channel filled with the gene-retaining carrier, and further transported to a reaction chamber in which the gene is detected. Then, the presence of the analysis gene of interest is detected. An example is specifically described below.

An analytic chip, that had been refrigerated or frozen, and which has built-in a washing solution I storage chamber 340, a washing solution II storage chamber 350, a washing solution III storage chamber 360, an eluting solution storage chamber 370, a gene-amplification reagent I storage chamber 380, and a gene-amplification reagent II storage chamber 390, which respectively contain six kinds of reagent, i.e. a washing solution I, a washing solution II, a washing solution III, a gene eluting solution, a gene-amplification reagent I and a gene-amplification reagent II, is thawed at room temperature. By providing a user with pre-embedded reagent for only one detection in the analytic chip 300, the analytic chip 300 can be rendered as a single-use chip which does not waste any reagent, thereby improving cost effectiveness.

The user can obviate the work required to deliver the reagents into each reagent storage chamber, which can not only shorten time, but can prevent contamination. Furthermore, by providing the user with the analytic chip 300 in a frozen state, if the user stores the analytic chip 300 frozen at 0° C., the activity of the reagent can be maintained for one month. Also, by storing frozen at −20° C., the activity of the reagent can be maintained for half a year or longer. By providing the user with a disposable analytic chip 300 pre-embedded with reagent for one detection in a refrigerated or frozen state, a simple analysis environment can be created (Step 101).

After the analytic chip 300 has been thawed, approximately 100 μL of the sampling chip 200 treating solution is sent to the sample injection port 310 of the analytic chip 300 (Step 102).

A cover is put over the sample injection port 310 to block the aperture. As the cover, a thin resin sheet of the same material as the analytic chip 300 is preferable. Since adhesion between the resins is good, and the resin is low cost, this is suitable for a single-use chip. While the step of covering the sample injection port 310 may be conducted manually, it is more preferable to have on the analyzer 400 side a mechanism which covers the sample injection port 310 (Step 103)

The fore lid 401 of the analyzer 400 is opened, and the analytic chip 300 is set into the analyzer 400 by following the chip guide provided on the fore lid 401, and the fore lid 401 of the analyzer 400 is then closed. By doing this, the analytic chip 300 is fixed onto the substrate 410, and the chip port and the device internal channel 402 are in communication with each other. While the analytic chip 300 may be placed either on its side or in an upright manner, the example described here is explained for when it is in an upright manner (Step 104).

Subsequently, by switching the valve 430 in the analyzer 400, a fluid is run from the pump 440 only to the chip port 311 (ports 311, 331: open; other ports: closed). The fluid used here may be any kind of fluid such as air or nitrogen as long as it does not deteriorate the activity of the reagent when contacted with the reagent. The reagent in the sample reservoir 315 moves to the gene extraction area 320. As a result of the action of the chaotropic ions in the sample, the bacteria gene in the sample bind to the gene binding carrier filling the gene extraction area 320. To promote the binding between the bacteria gene and the gene binding carrier, the sample is preferably passed through the gene extraction area 320 for 10 minutes or more. The sample which has passed through the gene extraction area 320 accumulates in the waste solution chamber 330. The gas used for transportation is discharged from the chip port 331. It is noted that by placing the chip in an upright manner, the sample can be prevented from leaking from the chip port 331 (Step 105).

To perform gene detection stably and with high accuracy, the gene and the gene binding carrier must reliably bind. To make sure that the gene and the gene binding carrier reliably bind, it is necessary for the gene and the gene binding carrier to be in reliable contact for a certain duration or longer. As a method for reliably contacting the gene and the gene binding carrier for a certain duration or longer, transporting the sample in an intermittent manner is suitable. Specifically, it is preferable to transport the sample while repeatedly opening and closing the chip port 311. For example, by repeatedly conducting the operation of closing the sample port for 29 seconds then opening the sample port for 1 second between 20 and 40 times, the sample liquid is reliably passed through the gene binding carrier over 10 to 20 minutes, thereby enabling the gene and the gene binding carrier to reliably bind. Since this only involves controlling the opening and closing of the chip port 311, the operation does not require a complex fluid control mechanism. It is thus possible to execute the operation with a simple device.

Subsequently, by switching the valve 430 in the analyzer 400, closing the chip port 311 and opening the chip port 341, a fluid is run from the pump 440 only to the chip port 341 (ports 331, 341: open; other ports: closed). 200 μL of the washing solution I in the washing solution I storage chamber 340 is transported to the gene extraction area 320 by the fluid. Preferred examples of the washing solution I include chaotropic ions such as guanidine thiocyanate, guanidine hydrochlorate, sodium iodide, potassium bromide or the like. Proteins remaining in the gene extraction area 320 as a result of the washing solution I are removed. The washing solution I which has passed through the gene extraction area 320 accumulates in the waste solution chamber 330 (Step 106).

Subsequently, by switching the valve 430 in the analyzer 400, closing the port 341 and opening the washing solution II port 351, a fluid is run from the pump 440 only to the washing solution II port 351 (ports 331, 351: open; other ports: closed). 150 μL of the washing solution II in the washing solution II storage chamber 350 is transported to the gene extraction area 320 by the fluid. Preferred examples of the washing solution II include highly concentrated ethanol of 50% or greater, potassium acetate or the like. Chaotropic ions remaining in the gene extraction area 320 as a result of the washing solution II are removed. The washing solution II which has passed through the gene extraction area 320 accumulates in the waste solution chamber 330 (Step 107).

The valve 430 in the analyzer 400 is switched to close the port 351 and open the washing solution III port 361. A fluid runs from the pump 440 only to the washing solution III port 361 (ports 331, 361: open; other ports: closed). 80 μL of the washing solution III in the washing solution III storage chamber 360 is transported to the gene extraction area 320 by the fluid. Preferred examples of the washing solution III include ethanol having a lower concentration than the washing solution II, potassium acetate having a lower concentration than the washing solution II, or pure water. As a result of the washing solution III, substances which hinder gene amplification such as chaotropic ions and washing solution II (highly concentrated ethanol, potassium acetate etc.) remaining in the gene extraction area 320 can be removed to a high degree, thereby allowing stable and accurate gene detection. The washing solution III which has passed through the gene extraction area 320 accumulates in the waste solution chamber 330 (Step 108).

Subsequently, by switching the valve 430 in the analyzer 400, closing the ports 331 and 361, and opening the chip port 371 and the chip port 399, a fluid is run from the pump 440 only to the chip port 371 (ports 371, 399: open; other ports: closed). 10 μL of the eluting solution in the eluting solution storage chamber 360 is transported to the gene extraction area 320 by the fluid. As the eluting solution, sterilized distilled water, a buffer solution such as TRIS-EDTA and TRIS-acetate or the like can be used. The gene trapped by this eluting solution on the gene binding carrier in the gene extraction area 320 are eluted. The eluted gene is transported to the reaction chamber 395 (Step 109).

To perform gene detection stably and with high accuracy, the gene bound to the gene binding carrier must be reliably eluted and transported to the reaction chamber 395. To make sure that the gene bound to the gene binding carrier is reliably eluted, it is necessary for the gene bound to the gene binding carrier and the eluent to be in reliable contact for a certain duration or longer. As a method for reliably contacting the gene bound to the gene binding carrier and the eluent for a certain duration or longer, transporting the eluent in an intermittent manner is preferred. Specifically, this method transports the eluent while repeatedly opening and closing the chip port 371. For example, by repeatedly conducting the operation of closing the chip port 371 for 29 seconds then opening the chip port 371 for 1 second between 20 and 40 times, the eluent is reliably passed through the gene binding carrier over 10 to 20 minutes, thereby enabling the gene bound to the gene binding carrier and the eluent to reliably bind. Further, since this only involves controlling the opening and closing of the chip port 371, the operation does not require a complex fluid control mechanism. It is thus possible to execute the operation with a simple device.

Subsequently, by switching the valve 430 in the analyzer 400, closing the port 371, and opening the chip port 381, a fluid is run from the pump 440 only to the chip port 381 (ports 381, 399: open; other ports: closed). 10 μL of the gene-amplification reagent I in the gene-amplification reagent I storage chamber 380 is transported to the reaction chamber 395 by the fluid. The gene-amplification reagent I is composed of four types of dNTP (dATP, dCTP, dGTP, dTTP), a buffer (Tris-HCl, KCl, MgCl₂), and a primer. The gas used for transportation is discharged from the chip port 399 (Step 110).

Subsequently, by switching the valve 430 in the analyzer 400, closing the port 381, and opening the chip port 391, a fluid is run from the pump 440 only to the chip port 391 (ports 391, 399: open; other ports: closed). 30 μL of the gene-amplification reagent II in the gene-amplification reagent II storage chamber 390 is transported to the reaction chamber 395 by the fluid. The gene-amplification reagent II is composed of DNA synthetases (such as Taq DNA polymerase, Tth DNA polymerase, Vent DNA polymerase, and thermosequenase), and a fluorescent dye (Step 111).

In accordance with the above procedures, the bacteria gene and two kinds of gene-amplification reagent have been introduced into the reaction chamber 395 of the analytic chip 300. Then, to amplify and detect the bacteria gene in the reaction chamber 395, the temperature control mechanism 415 is activated, and a temperature cycle is applied so that the temperature of the reaction chamber 395 moves back and forth between the following two predetermined values (Step 112).

As an example of temperature cycle, the following may be performed:

[90 to 95° C. for 10 to 30 seconds [symbol] 65 to 70° C. for 10 to 30 seconds]×30 to 45 times

As a preferred example, the following temperature cycle may be performed:

[94° C. for 30 seconds [symbol] 68° C. for 30 seconds]×45 times

While the temperature cycle is performed, an excitation light is irradiated onto the reaction chamber 395 from the light source 450. The gene, if it has a fluorescent dye intercalated into the inside of the double strand, transfers the energy of the absorbed light of the light source 450 to the fluorescent dye (energy transfer). As a result, the fluorescent dye is excited and emits fluorescence. Thus, when the gene of interest is present in the sample, the amount of fluorescence emitted increases as the gene is amplified. Therefore, by monitoring the amount of fluorescence in the reaction chamber 395 by the photodetector 181 during the temperature cycle, the presence or absence of the gene of interest can be detected in real time as shown in FIG. 7. Placing the analytic chip 300 in the analyzer 400 in an upright manner has the advantage of preventing a decrease in the detection sensitivity, because the reaction chamber 395 side face which detects the fluorescence does not cloud up even if a part of the reaction substance evaporates during the temperature cycle to cause vapor to accumulate at an upper section of the reaction chamber 395 (Step 113).

After analysis is completed, the analytic chip 300 is removed from the analyzer 400, and discarded. This obviates the need for post-treatment of the samples and the reagents and the need for a washing procedure of the reaction detection section, thereby enabling simple and rapid analysis to be provided (Step 114).

It should be further understood by those skilled in the art that although the foregoing description has been made on embodiments of the invention, the invention is not limited thereto and various changes and modifications may be made without departing from the spirit of the invention and the scope of the appended claims.

By using a sampling chip and analytic chip in combination with a sampler and analyzer, the steps from bacteria sample to gene extraction can be automated in two kinds of small chip. Since no manual operation is involved in the bacteria spore treatment or the gene extraction step, any person can safely conduct the analysis. Further, since the interior of the compact chip is free from mechanical parts such as valves, a chip can be provided which is suitable for single-use applications. Furthermore, as a result of miniaturization of the volume of the reaction chamber and the channels by microfabrication, it is possible to obtain such advantages as reduction in the amount of reagents and in cost as well as rapid temperature control, rapid mixing, and homogeneous reaction. Moreover, since the reagent for just one detection is pre-embedded in a disposable analytic chip and the analytic chip is provided to the user in a refrigerated or frozen state, extremely simple and fast gene detection can be attained. Further, such as chip can be disposed of with the reagents after the analysis is completed.

### Advantages of the Invention

According to the present invention, since a weir is provided in at least either the sample reservoir or the reagent storage chamber on an analytic chip on which treatments from gene extraction to detection of are conducted, there is no accidental leakage, accuracy is good, and it is possible to detect a minute creature in a stable manner.

1. A minute creature detection system in which, a sampling chip is placed in a sampler to sample minute creatures on a sampling material and is then removed from the sampler and placed in an analyzer, and the sampling chip and a part of the solution treated by the sampling chip are moved to an analytic chip, and the treatments from gene extraction to detection are conducted on the analytic chip by liquid transport means of the analyzer, the analytic chip comprising:
a sample reservoir, a gene extraction area filled with a gene binding carrier, a waste solution chamber filled with absorbent, a washing solution storage chamber which stores washing solution, an eluenting solution storage chamber which stores a gene eluenting solution, a gene-amplification reagent storage chamber which stores a gene-amplification reagent, and a reaction chamber which amplifies and detects the gene, each of which is formed by a channel, and a weir having a channel in at least either the sample reservoir or the reagent storage chamber which contracts and expands.

2. The minute creature detection system according to claim 1, wherein taking an expanding angle as θ₂, a contact angle α between the sample or the reagent and the analytic chip is represented by α−θ₂>0.

3. The minute creature detection system according to claim 1, wherein contraction of the weir channel width is not more than ¼ and not less than 1/10 of the channel width.

4. The minute creature detection system according to claim 1, wherein the channel in communication with the gene-amplification reagent storage chamber is provided at a location higher than the gene-amplification reagent storage chamber.

5. The minute creature detection system according to claim 1, wherein the contracting angle θ₁ is 10° or more to 80° C. or less.

6. The minute creature detection system according to claim 1, wherein the expanding angle θ₂ is 5° or more to 80° C. or less.

7. The minute creature detection system according to claim 1, wherein the sample is intermittently fed to the gene extraction area.

8. An analytic chip wherein a part of the solution treated by a sampling chip of a minute creature sampled on a sampling material provided in a sampler is moved, and the treatments from gene extraction to detection of are conducted by liquid transport means of an analyzer, the analytic chip comprising:
a sample reservoir, a gene extraction area filled with a gene binding carrier, a waste solution chamber filled with absorbent, a washing solution storage chamber which stores washing solution, an eluenting solution storage chamber which stores a gene eluenting solution, a gene-amplification reagent storage chamber which stores a gene-amplification reagent, and a reaction chamber which amplifies and detects the gene, each of which is formed by a channel, a sample injection port open to a front face, a chip port open to a rear face, and a weir having a channel in at least either the sample reservoir or the reagent storage chamber which contracts and expands.
